(19) United States
(12) Patent Application Publication
(10) Pub. No.: US 2024/0229121 A1
(43) Pub. Date: Jul. 11, 2024

(54) MICRO-RNA DETECTION METHOD AND KIT

(71) Applicant: ZHEJIANG LAB, HANGZHOU, Zhejiang Province (CN)

(72) Inventors: PEIXIANG MA, HANGZHOU, ZHEJIANG PROVINCE (CN); MINGTIAN ZHONG, HANGZHOU, ZHEJIANG PROVINCE (CN); LU DANG, HANGZHOU, ZHEJIANG PROVINCE (CN); BAICHENG HUANG, HANGZHOU, ZHEJIANG PROVINCE (CN)

(21) Appl. No.: 18/561,284

(22) PCT Filed: Mar. 21, 2023
(86) PCT No.: PCT/CN2023/082682

§ 371 Date: Nov. 15, 2023

(30) Foreign Application Priority Data
Apr. 21, 2022 (CN) 202210420863.9

(51) Int. Cl. C12Q 1/6851; C12Q 1/6823

(52) U.S. Cl. CPC C12Q 1/6851; C12Q 1/6823; C12Q 2600/178

(57) ABSTRACT

The present invention discloses a microRNA detection method and kit for rapid detection of microRNA nucleic acids in biological and environmental samples, including microRNA tailing and a Cpf1 detection system suitable for rapid detection. The present invention is the first to use a combination of microRNA tailing and Cpf1 detection to detect microRNAs, with the advantages of high sensitivity, strong specificity, short time consumption, high throughput, direct interpretation by the naked eye, no dependence on large-scale experimental equipment and the like. These advantages make the detection method developed by the present invention convenient for rapid detection, and identification and diagnosis of microRNAs in biological and environmental samples at a clinical front line.

### TECHNICAL FIELD

The present invention belongs to the field of biotechnology, and in particular relates to a microRNA detection method and kit.

### BACKGROUND TECHNOLOGY

MicroRNAs (miRNAs) are small non-coding RNAs composed of approximately 22 nucleotides, playing an important role in regulating the expression of protein-coding genes and the pathogenesis of tumors. Recent studies have shown that abnormal expression of miRNAs can serve as an effective biomarker for the diagnosis, progression, and monitoring of human diseases. Changes in extracellular circulating miRNA levels in serum, plasma, saliva, urine, and other body fluids can be used as novel non-invasive disease biomarkers. The traditional techniques used for detecting miRNAs include Northern Blot, quantitative reverse transcription PCR (RT-qPCR), second-generation sequencing, in situ hybridization, and microarray-based hybridization. However, these technologies are time-consuming and costly, so their drawbacks limit their accessibility for use. Therefore, it is necessary to develop a fast, economical, stable, sensitive, and specific miRNA detection technology.

CRISPR-Cas systems were originally derived from the RNA-guided adaptive immune system of bacteria, and used to resist the nucleic acid components of invading bacteria and archaea. Various emerging CRISPR-Cas systems provide powerful tools for gene regulation and genome engineering. In particular, the parallel cleavage activity of Cpf1 can be developed as a diagnostic tool for highly specific nucleic acid detection, and can even differentiate single nucleotide polymorphisms (SNPs). The main challenges of miRNA quantitative detection are the short sequence length, high sequence similarity among family members, and sequence length variation of miRNAs. Due to shorter miRNA sequences and fewer PAMs in miRNA sequences, it is difficult to directly detect with CRISPR. Therefore, there are issues with the detection of miRNAs, such as long testing time, low efficiency, insufficient specificity and sensitivity, and high testing costs.

### Explanation of Terms

The term crRNA refers to CRISPR RNA, which is a short RNA that guides Cpf1 to bind to the target DNA sequence. The term CRISPR refers to clustered, regularly interspaced short palindromic repeats, which is the immune system of many prokaryotes.

The term Cas protein refers to a CRISPR-associated protein, which is a related protein in the CRISPR system.

The term Cpf1 (also known as Cas12a) refers to a crRNA-dependent endonuclease, which is a type V enzyme in the CRISPR system classification. The term PAM refers to a protospacer-adjacent motif, which is necessary for Cpf1 cleavage. The PAM of FnCpf1 is a TTN sequence, and the PAM of LbCpf1 is a TTTN sequence.

The term RT-RPA refers to reverse transcription isothermal amplification, also known as reverse transcription-recombinase polymerase amplification.

### SUMMARY OF INVENTION

The technical problem to be solved by the present invention is to overcome the defects of high costs, long detection time, low detection efficiency, and low specificity and sensitivity in detecting miRNA nucleic acids in existing technologies, and the present invention provides a detection method and kit with combined use of poly-adenine (PolyA) polymerase and Cpf1, which solves the problems of difficulty in miRNA amplification and difficulty in accurate detection due to high sequence similarity.

The present invention provides a microRNA detection method, including following steps:
S1: poly-adenine (PolyA) tailing, adding a poly-adenine (PolyA) sequence of at least two adenines to a microRNA of a sample; S2: cDNA synthesis, synthesizing a cDNA based on a tailed microRNA; S3: DNA amplification, amplifying the synthesized cDNA; S4: Cpf1 detection, recognizing the cDNA by Cpf1, and activating Cpf1 to cleave a probe for detection.

The present invention also provides a microRNA detection kit, which includes PolyA tailing and a Cpf1 detection system.

As a preferred choice, the poly-adenine (PolyA) tailing includes a poly-adenine (PolyA) polymerase, which is a codon-optimized protein and a mutated protein containing a functional domain of the polymerase. Preferably, the Cpf1 detection system includes a Cpf1 protein and/or a nucleic acid probe.

Preferably, the Cpf1 protein is a codon-optimized Cpf1 protein, prokaryotic codon optimization is performed on various sources of cpf1 protein nucleic acid sequences to obtain sequences, which is constructed into a pET28a expression vector for low-temperature induced soluble protein expression, and a target protein is obtained through affinity purification and molecular sieve purification.

Preferably, the nucleic acid probe is a single-stranded DNA and can be used for fluorescence detection.

Preferably, the nucleic acid probe is a fluorescence-labeled single-stranded DNA, with a fluorescence group at one end and a fluorescence quenching group at the other end. Preferably, the nucleic acid probe is a single-stranded DNA, with a fluorescence group at a 5′ end and a fluorescence quenching group at a 3′ end. Preferably, the fluorescent group is 6-FAM, TET, CY3, CY5, or ROX, the fluorescence quenching group is BHQ1, BHQ2, or BHQ3, and a sequence of the single-stranded DNA is TTTATTT.

Cpf1 can recognize DNA nucleic acid sequences under the guidance of RNAs, but the miRNA sequence is an RNA and cannot be directly recognized. Moreover, the RNA sequence is relatively short, with a length of only about 20 bases, and most of them do not have PAM recognition sequences that Cpf1 can recognize. The present invention creatively combines PolyA polymerase to solve three difficulties in detecting miRNAs by Cpf1: firstly, by using the PolyA sequence, after a miRNA is transcribed into a DNA, a TTT recognizable sequence is added, without the need to use the PAM sequence inside the miRNA, which can be used for any miRNA, with better versatility; secondly, it is transcribed into a DNA and can be highly specifically recognized by Cpf1; and thirdly, after transcription into a DNA, it can be more stable and less prone to degradation than a RNA. Therefore, this method can improve the signal strength of Cpf1 detection, which can be used to enhance the signal of Cpf1-mediated in vitro detection to reduce detection time and improve detection efficiency.

In the present invention, once a target nucleic acid, crRNA, and Cpf1 protein form a ternary complex, the complex will cleave other single-stranded DNA molecules in the system. A crRNA targeting a target nucleic acid is designed; the crRNA and Cpf1 protein are added to the detection system; when the target DNA is present, Cpf1 forms a ternary complex with the crRNA and target DNA, and at the same time, this complex exercises its parallel cleavage activity and cleaves single-stranded DNA labeled with fluorescence signals (with luminescent and quenching groups connected at both ends, which can emit fluorescence after being cut off), thereby emitting fluorescence. Therefore, by detecting fluorescence, it can be determined whether the target DNA molecule is present in the system to be tested.

In the present invention, a clinical sample usually can be inactivated to release nucleic acids from the sample to be tested. Under isothermal conditions, reverse transcription (RT) is performed on the miRNA of a sample to obtain a DNA, and recombinase polymerase amplification (RPA) is performed. The Cpf1-crRNA complex binds to and cleaves a target dsDNA, which activates the trans-cleavage of a ssDNA. The fluorescent reporter molecule coupled with the ssDNA generates a fluorescence signal during cleavage.

In the present invention, the nucleic acid probe can generally also be referred to as a fluorescence-quenching single-stranded DNA reporting system, which generally contains fluorescence and quenching groups. In a complete state, fluorescence is quenched by the quenching group, and after being cut during the reaction process, fluorescence is emitted. On the basis of complying with common knowledge in the field, the above preferred conditions can be arbitrarily combined to obtain various preferred examples of the present invention. The reagents and raw materials used in the present invention are commercially available.

The beneficial effects of the present invention are as follows:

PolyA tailing can solve the problem of short miRNA length and difficulty in amplification, and can be used to expand miRNA sequences and promote amplification; at the same time, to address the problem of Cpf1 being limited by PAMs and having fewer nucleic acid sequences to detect, poly-adenine (PolyA) can add universal PAM sequences to miRNAs, allowing this method to cover all microRNAs. By integrating the CRISPR-Cpf1 system, the specificity of Cpf1 can be utilized to solve the problem of sequence similarity among miRNA family members, thus enabling the identification of target nucleic acid sequences amplified from miRNAs. This system improves the stability of miRNA samples, reduces testing time and costs, and improves reproducibility. As demonstrated in the present invention, PolyA-CRISPR/Cpf1 can develop portable, accurate, and cost-effective real-time diagnostic systems.

When using the PolyA-Cpf1 kit of the present invention to detect miRNA nucleic acids, the signal strength during detection can be significantly improved, thereby reducing detection time and improving detection efficiency, with high sensitivity, high specificity, high accuracy, visual detection (can be visually detected directly with the naked eye under fluorescent lamps), low costs, no need for complex large experimental equipment, and easy operation. The kit and detection method of the present invention are made more suitable for rapid detection and identification diagnosis in basic experiments and clinical front lines. In a preferred embodiment of the present invention, the detection sensitivity for detecting viral DNA samples is miRNAs at a concentration of 1E-15 M.

### BRIEF DESCRIPTION OF THE DRAWINGS

FIG. 1 is a schematic diagram of a method for rapid detection of a miRNA by PolyA-Cpf1;

FIG. 2 is a schematic diagram of a coverage ratio of a miRNA detected by PolyA-Cpf1;

FIG. 3 is a schematic diagram 1 of a crRNA design for PolyA-Cpf1 detection of a miRNA;

FIG. 4 is a schematic diagram 2 of a crRNA design for PolyA-Cpf1 detection of a miRNA;

FIG. 5 is an experimental validation diagram for PolyA-Cpf1 detection of a miRNA;

FIG. 6 shows crRNA validation of miR-299;

FIG. 7 shows crRNA validation of miR-335;

FIG. 8 shows isothermal amplification primer validation;

FIG. 9 shows sensitivity analysis of PolyA-Cpf1 detection;

FIG. 10 is an experimental validation diagram for PolyA-Cpf1 detection of miR-21 in different cells;

FIG. 11 shows experimental validation for PolyA-Cpf1 detection of different miRNAs from different cell sources; and

FIG. 12 shows specificity of PolyA-Cpf1 detection, which can distinguish miRNAs of let7a with single base differences.

### DETAILED DESCRIPTION OF THE EMBODIMENTS

The following will further elaborate on the present invention in conjunction with specific embodiments. It should be understood that these embodiments are only used to illustrate the present invention and not to limit its scope. In addition, it should be understood that after reading the content taught in the present invention, those skilled in the art can make various changes or modifications to the present invention, and these equivalent forms also fall within the scope of the claims attached to this application.

The experimental methods without specific conditions specified in the following embodiments shall be selected according to conventional methods and conditions, or according to products' instructions. In the present invention, the RPA amplification kit TwistAmp® Basic kit is purchased from TwistAmp company; the CRRNA in vitro transcription box MEGAshortscript T7 Transcription Kit and purification box MEGAclear Kit are purchased from Ambion company; conventional reagents such as Tris-Base, BSA, NaCl, Tris-HCl, MgSO₄, BSA, and glycerol are purchased from Thermo Fisher; the synthesis of the nucleic acid fragments, ssDNA probes, and RNAs used for detection is completed by Nanjing Jinsirui company; the present invention uses a rapid nucleic acid releasing agent purchased from Vazyme company to obtain pre-processed nucleic acids.

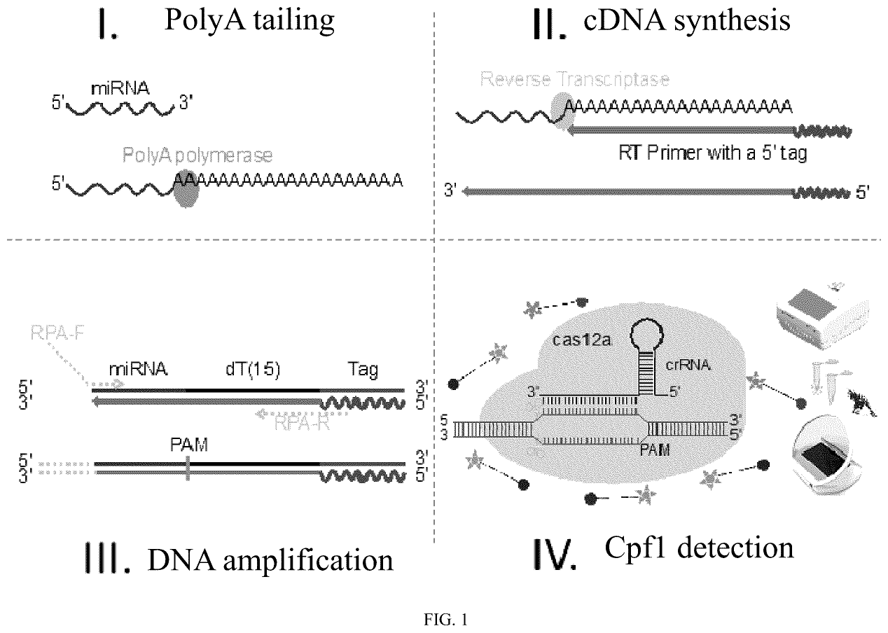

The overall technical schematic diagram of the present invention is shown in FIG. 1, including the following four major parts:
S1: poly-adenine tailing, adding a poly-adenine sequence of at least two adenines to a microRNA of a sample;
S2: cDNA synthesis, synthesizing a cDNA based on a tailed microRNA;
S3: DNA amplification, amplifying the synthesized cDNA;
S4: Cpf1 detection, recognizing the cDNA by Cpf1, and activating Cpf1 to cleave a probe for detection.

A microRNA detection kit includes poly-adenine tailing and a Cpf1 detection system, where the poly-adenine tailing includes a poly-adenine polymerase which is a codon-optimized protein and a mutated protein containing a functional domain of the polymerase, and the Cpf1 detection system includes a Cpf1 protein and/or a nucleic acid probe, where the Cpf1 protein is a codon-optimized Cpf1 protein, the nucleic acid probe is a single-stranded DNA, the nucleic acid probe is a fluorescence-labeled single-stranded DNA with a fluorescence group at one end and a fluorescence quenching group at the other end, and the nucleic acid probe is a single-stranded DNA with a fluorescence group at the 5′ end and a fluorescence quenching group at the 3′ end, where the fluorescence group is 6-FAM, TET, CY3, CY5, or ROX, and the fluorescence quenching group is BHQ1, BHQ2, or BHQ3, and a sequence of the single-stranded DNA is TTTATTT.

### Embodiment 1: PolyA-Cpf1 Detection of miRNAs

Validation of a Method for PolyA-Cpf1 Detection of a miRNA.

The LbCpf1 gene in this embodiment was optimized for codons (amino acid sequence as shown in SEQ ID NO.1 and gene sequence as shown in SEQ ID NO.2), cloned into the pet28a plasmid (completed by Nanjing Jinsirui company), expressed in Escherichia coli, purified, and used for detection experiments.

The experimental system for PolyA tailing was a 20 μl system, with the addition of 15 μL RNA (total amount of approximately 1-10 μg), 2 μL 10× Poly(A) Polymerase Reaction Buffer solution, 2 μL ATP (10 mM), and 1 μL Poly(A) Polymerase.

Preparation of nucleic acids. CrRNAs and miRNAs were synthesized by GenScript (Nanjing. China), and the core sequences used in this study are listed in Tables 1 and 2 (supporting information). For miRNA reverse transcription, a one-step miRNA cDNA synthesis kit (HaiGene) was used to synthesize cDNAs and minor modifications were made according to the manufacturer's protocol. The reverse transcription of miRNAs was optimized: 5 μL reverse transcription reaction (including 1.25 μL reverse transcription buffer, 0.5 μL reverse transcription primers and miRNAs) was performed at a constant temperature of 37° C. for 60 min, and then terminated after reacting at a constant temperature of 95° C. for 5 min.

Isothermal amplification. The isothermal amplification of miRNA cDNAs was carried out using a commercial RPA kit (GenDx) according to the manufacturer's instructions. 25 μL isothermal amplification reaction includes 5 μL reverse transcript product, 10 μL reaction solution, 0.5 μM forward primer, 0.5 μM reverse primer, followed by 1 μL activator, which reacts at a constant temperature of 37° C. for 20 minutes. The primer sequences used in this study are listed in Table 3.

CRISPR/Cas12a-based fluorescence detection. CrRNA consists of a 21 nt region that interacts with Cas12a and a 23 nt programmable guide region (called a spacer) for target DNA recognition. A single-stranded DNA reporter gene (ssDNA-FQ) labeled with FAM and BHQ1 was synthesized by GenScript (Nanjing, China). 200 ng purified Cas12a. 25 pM ssDNA-FQ, 1 μM crRNA, and 2 μL sample were used for detection, with a final volume of 20 μL. The reaction was incubated at 37° C. In the detection system, once Cas12a was activated by the target sequence, ssDNA-FQ would be cleaved. A full-wavelength microplate reader was used to measure the fluorescence of the detection reaction, with an excitation wavelength of 485 nm and an emission wavelength of 520 nm. The signal was recorded every 1 minute for 1 hour. A mobile phone camera was used to take photos under excitation light of 485 nm.

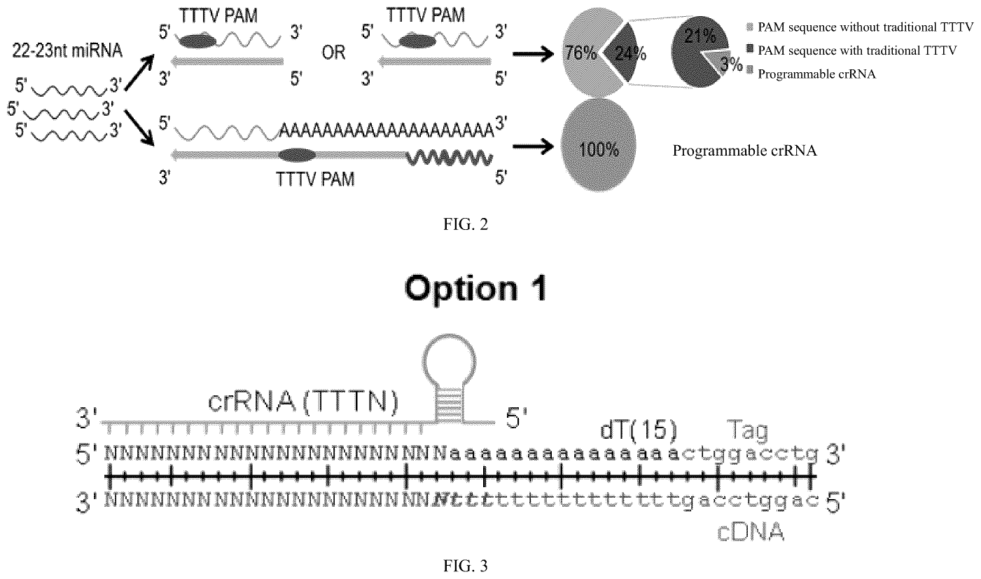

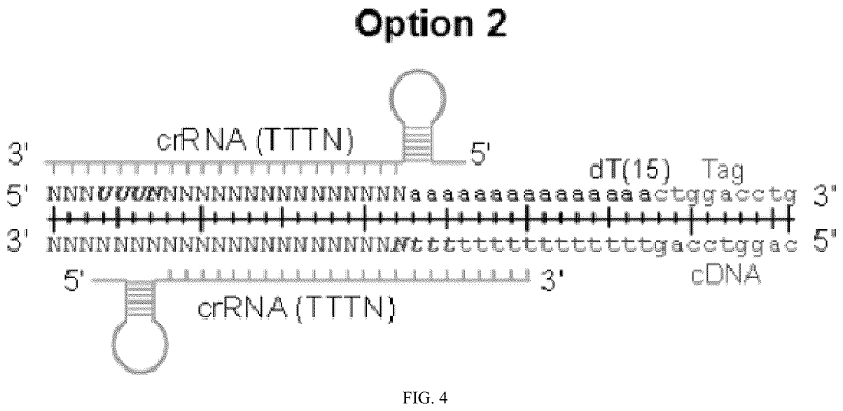

As shown in FIG. 2, the PAM sequences contained in all known miRNAs were analyzed, and PolyA tailing can cover all miRNA detections. As shown in FIG. 3, the crRNA design method 1 can be selected after PolyA tailing; as shown in FIG. 4, the crRNA design method 2 can be selected after Poly A tailing.

### Embodiment 2: Detection of Nucleic Acid Fragments in Cell Samples

### Nucleic Acid Preparation.

Cell culture and miRNA extraction. SNU-449, PC-9, U87MG, U251, Jurkat and 16HBE were all from the Chinese Academy of Sciences (Shanghai). All cells except Jurkat and 16HBE cells were cultured in Dulbecco modified Eagle's medium (DMEM) containing 10% fetal bovine serum (FBS). Jurkat and 16HBE were cultured in RPMI 1640 medium containing 10% fetal bovine serum. When the cell density reached 80-90%, cells were collected and miRNAs were extracted using a commercial MiPure cell/Tissue miRNA kit (Vazyme). The total miRNA concentration was measured after purification using a Nanodrop 2000 instrument. The extract was stored at −80° C.

Preparation of nucleic acids. CrRNAs were synthesized by GenScript (Nanjing, China), and the core sequences used in this study are listed in Tables 1 and 2. For miRNA reverse transcription, a one-step miRNA cDNA synthesis kit (HaiGene) was used to synthesize cDNAs and minor modifications were made according to the manufacturer's method. The reverse transcription of miRNAs was optimized: 5 μL reverse transcription reaction (including 1.25 μL RT solution, 0.5 μL RT primers and miRNA quantity) was performed in a 37° C. incubator for 60 min, and then in a 95° C. incubator for 5 min.

Isothermal amplification. The isothermal amplification of miRNA cDNAs was carried out using a commercial RPA kit (GenDx) according to the manufacturer's instructions. 25 μL isothermal amplification reaction includes 5 μL reverse transcript product, 10 μL reaction solution, 0.5 μM forward primer, 0.5 μM reverse primer, followed by 1 μL activator solution, which was performed for constant temperature amplification, with amplification at 37° C. for 20 minutes.

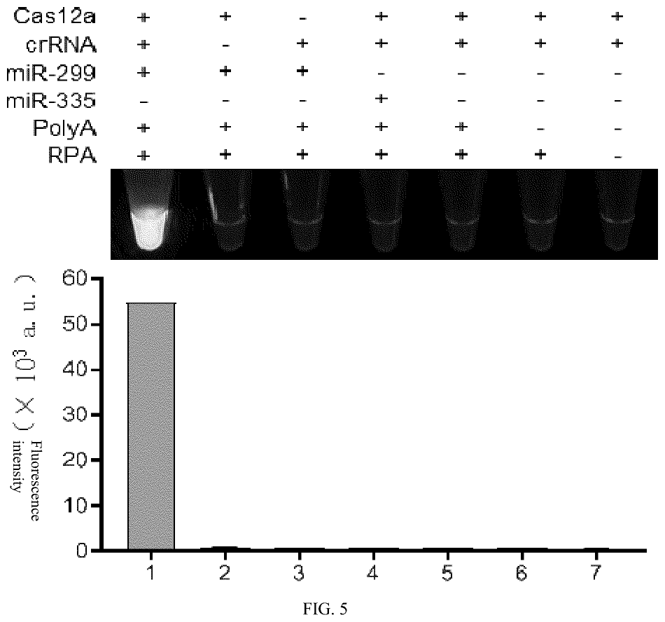

As shown in FIG. 5, for miR-299, PolyA, amplification, and Cas12a detection were used to confirm the feasibility of this scheme.

TABLE 1
CRRNA sequences

| crRNA | crRNA sequence and | | |
|---|---|---|---|
| Name | PAM (5′ to 3′) | Target | Direction |
| miR-299- | TTTTATGTATGTGGGACG | miR-299 | − |
| crRNA1 | GTAAACCAG | | |
| miR-299- | TTTATGTATGTGGGACGG | miR-299 | − |
| crRNA2 | TAAACCAGG | | |
| miR-299- | TTTACCGTCCCACATACA | miR-299 | + |
| crRNA3 | TAAAAAAAA | | |
| miR-335- | TTTTACATTTTTCGTTAT | miR-335 | − |
| crRNA4 | TGCTCTTGA | | |
| miR-335- | TTTACATTTTTCGTTATT | miR-335 | − |
| crRNA5 | GCTCTTGAG | | |
| let-7a- | TTTAACTATACAACCTAC | let-7a | − |
| crRNA | TACCTCA | | |
| miR-2 | TTTACAACATCAGTCTGA | miR-21 | − |
| | TAA | | |

TABLE 2
MicroRNA sequences

| miRNA name | Sequence (5′ to 3′) |
|---|---|
| miR-299-5p | UGGUUUACCGUCCCACAUACAU |
| miR-335-5p | UCAAGAGCAAUAACGAAAAAUGU |
| let-7a | UGAGGUAGUAGGUUGUAUAGUU |
| let-7b | UGAGGUAGUAGGUUGUGUGGUU |
| let-7e | UGAGGUAGGAGGUUGUAUAGUU |
| miR-122 | UGGAGUGUGACAAUGGUGUUUG |

### Embodiment 3: Optimization of crRNAs

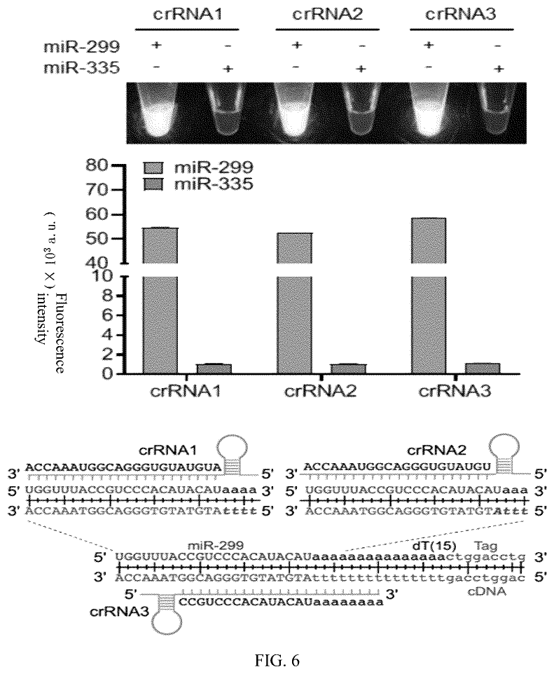

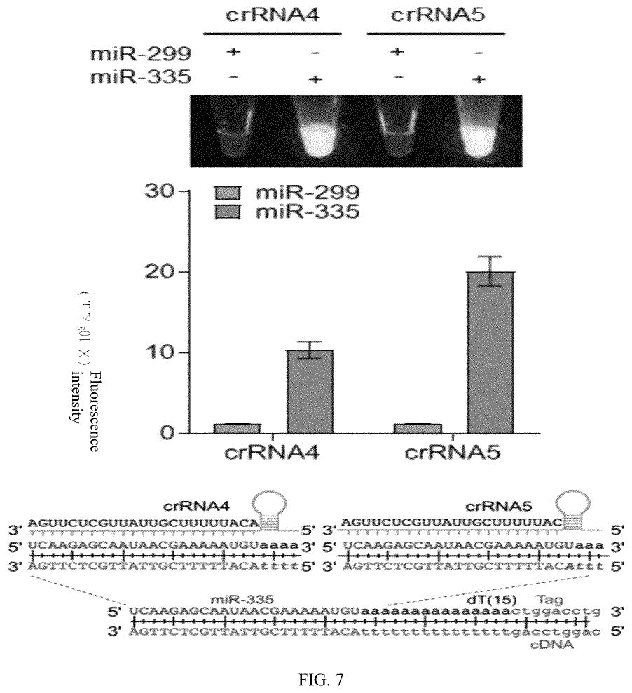

In this embodiment, crRNA1, 2, 3, 4, and 5 were designed to detect whether crRNAs can mediate efficient recognition, and the specific sequences are listed in Table 1. The reaction was performed according to the reaction conditions in Embodiment 1. After PolyA tailing, cDNA synthesis, and DNA amplification, the reaction was performed using cpf1. The results are shown in FIGS. 6 and 7.

Conclusion: As shown in FIGS. 6 and 7, the crRNAs constructed using PAMs derived from PolyA can efficiently recognize the microRNAs to be tested, confirming that crRNAs can be designed using the PolyA ending method.

### Embodiment 4: Optimization of Detection

DNA amplification is a crucial step in detection, and it is necessary to optimize amplification primers to achieve efficient detection. For each microRNA to be tested, multiple pairs of forward and reverse primers needed to be designed to compare the effectiveness of amplification and the efficiency of detection. The sequences of amplification primers are listed in Table 3.

TABLE 3
Amplified primers

| Primer name | Sequence (5′ to 3′) |
|---|---|
| miR-299-RPA-F1 | TCTACGTCGGGGTGGTTT |
| miR-299-RPA-F2 | TACGTCGGGGTGGTTTAC |
| miR-299-RPA-F3 | CGTCGGGGTGGTTTACCG |
| miR-335-RPA-F1 | TCTACGTCGGGGTCAAGA |
| miR-335-RPA-F2 | TACGTCGGGGTCAAGAGC |
| miR-335-RPA-F3 | CGTCGGGGTCAAGAGCAA |
| miRNA-RPA-R1 | CGCGCAGGTCCAGTTTTTTT |
| miRNA-RPA-R2 | CGCGCAGGTCCAGTTTTTTTTT |
| miRNA-RPA-R3 | CGCGCAGGTCCAGTTTTTTTTTTT |
| let-7a-RPA-F | TCTACGTCGGGGTGAGGT |
| let-7a-RPA-R | CGCGCAGGTCCAGTTTTTTT |
| miR-21-RPA-F | TACGTCGGGGTAGCTTAT |
| miR-21-RPA-R | CGCGCAGGTCCAGTTTTTTTTTTT |
| miR-21-PolyA-qPCR-F | GCAGTAGCTTATCAGACTGATG |
| miR-21-PolyA-qPCR-R | GGTCCAGTTTTTTTTTTTTTTTCAAC |

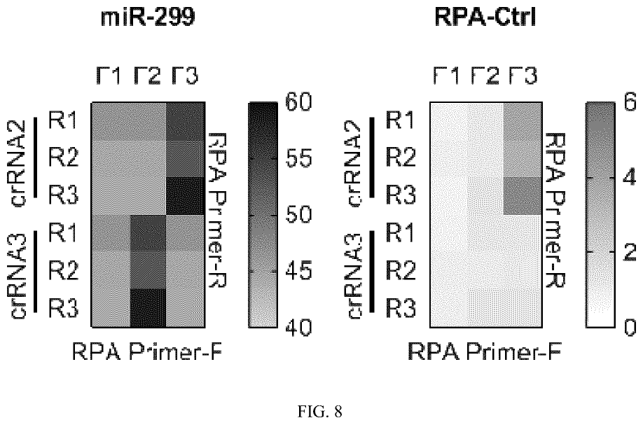

Conclusion: as shown in FIG. 8, F2R3 and F3R3 had the highest amplification efficiency for miR299.

### Embodiment 5: Sensitivity of Detection

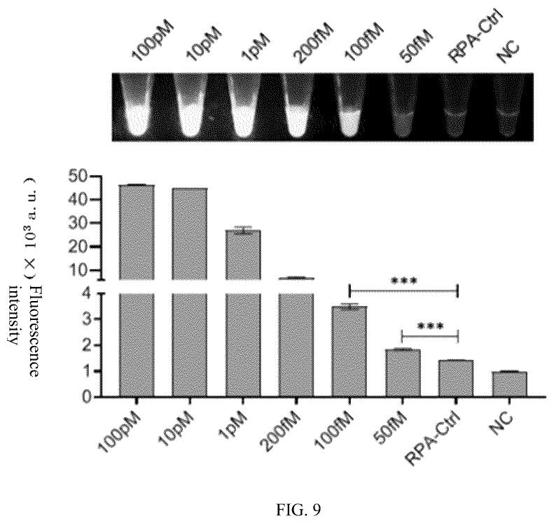

To determine the detection sensitivity of this system, we performed a series of gradient dilutions on miR-299 RNA, and performed the reaction according to the reaction conditions of the steps in Embodiment 1. After PolyA tailing, cDNA synthesis, and DNA amplification, the reaction was performed using cpf1. The results are shown in FIG. 9.

Conclusion: As shown in FIG. 9, a minimum of 50 fM miR-299 RNA can be detected.

### Embodiment 6: Scalability of Detection

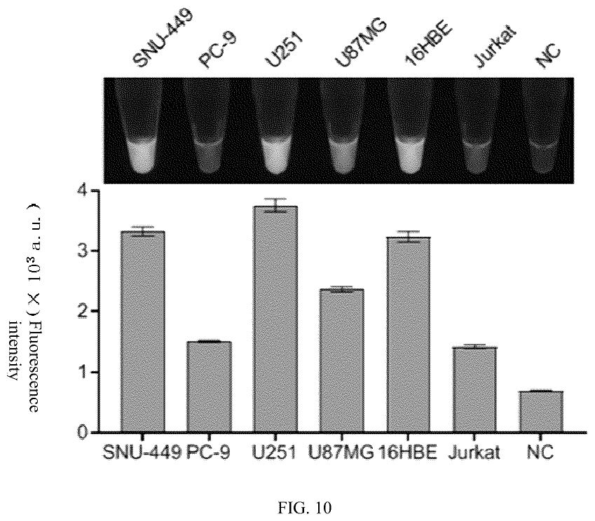

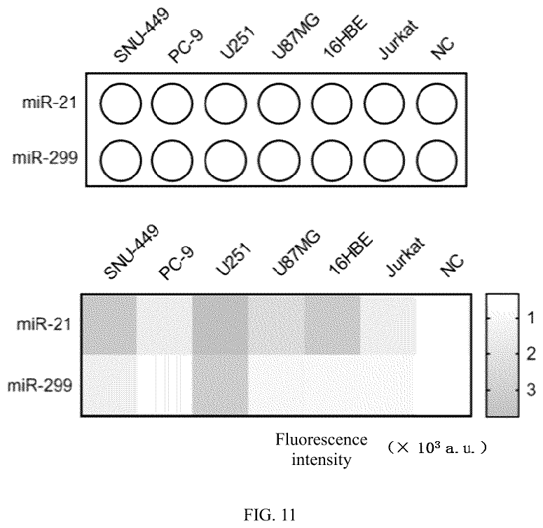

To determine the scalability of this system's detection, we conducted tests on cell lysates of miRNA-21 and miRNA299, as well as SNU-449, PC-9, U251, U87MG, 16HBE, Jurkat, and medium blank control (NC). After extracting the nucleic acid, the reaction was carried out according to the reaction conditions of the steps in Embodiment 1, and the core sequences used are listed in Tables 1 and 2. After PolyA tailing, cDNA synthesis, and DNA amplification, reaction detection was performed using cpf1. The results are shown in FIGS. 10 and 11.

### Conclusion

As shown in FIG. 10, this method can detect the content of miRNA-21 in different cells.

As shown in FIG. 11, this method can be changed to an array mode to detect the content of different miRNAs in various cells.

### Embodiment 7: Specificity of Detection

In order to determine the detection specificity of this system, we used the microRNAs of let7a, 7b, and 7e with similar sequences, with a minimum difference of only one base, and then performed the reaction according to the reaction conditions of the steps in Embodiment 1. After PolyA tailing, cDNA synthesis, and DNA amplification, the reaction was performed using cpf1.

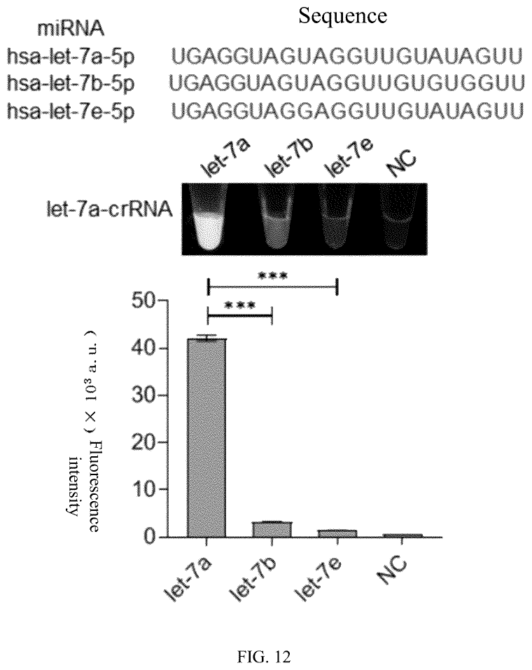

Conclusion: As shown in FIG. 12, this method can accurately distinguish microRNAs with one base difference and had high specificity.

The above is only a preferred specific implementation of the present invention, but the scope of protection of the present invention is not limited to this. Equivalent substitutions or changes based on the technical solutions and inventive concept of the present invention by any one skilled in the art within the technical scope disclosed by the present invention shall be covered within the scope of protection of the present invention.

1. A microRNA detection method, characterized in that the method is used for detection for non-disease diagnosis purposes, comprising following steps:
S1: poly-adenine tailing, adding a poly-adenine sequence of at least two adenines to a microRNA of a sample;
S2: cDNA synthesis, synthesizing a cDNA based on a tailed microRNA;
S3: DNA amplification, amplifying the synthesized cDNA;
S4: Cpf1 detection, recognizing the cDNA by Cpf1, and activating Cpf1 to cleave a probe for detection.

2. A microRNA detection kit, characterized in that the kit comprises a poly-adenine polymerase and a Cpf1 detection system; the Cpf1 detection system comprises a Cpf1 protein and a nucleic acid probe.

3. The microRNA detection kit according to claim 2, characterized in that the poly-adenine polymerase is a codon-optimized protein or a mutated protein containing a functional domain of the polymerase.

4. The microRNA detection kit according to claim 2, characterized in that the Cpf1 protein is a codon-optimized Cpf1 protein.

5. The microRNA detection kit according to claim 2, characterized in that the nucleic acid probe is a single-stranded DNA.

6. The microRNA detection kit according to claim 5, characterized in that the nucleic acid probe is a fluorescence-labeled single-stranded DNA, with a fluorescence group at one end and a fluorescence quenching group at the other end.

7. The microRNA detection kit according to claim 6, characterized in that the nucleic acid probe is a single-stranded DNA, with a fluorescence group at a 5′ end and a fluorescence quenching group at a 3′ end.

8. The microRNA detection kit according to claim 7, characterized in that the fluorescence group is 6-FAM, TET, CY3, CY5, or ROX, the fluorescence quenching group is BHQ1, BHQ2, or BHQ3, and a sequence of the single-stranded DNA is TTTATTT.


SEQUENCE LISTING (30 sequences)

SEQ ID NO: 1 - protein, 1301 aa, synthetic construct
MGPKKKRKVAAADYKDDDDKSRLEPGEKPYKCPECGKSFSQSGALTRHQRTHTRSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHENLYFQSHHHHHHHHHHHHH

SEQ ID NO: 2 - DNA, 3903 bp, synthetic construct
  source 1..3903: mol_type = other DNA
atgggtccaaagaagaagcgtaaggttgccgctgccgattacaaagatgatgatgataaatcgcgcttggagcccggcgaaaaaccatacaagtgccctgagtgcggcaaatcgttctcccagagcggtgctttgacccgtcaccaacgcactcatacccgttccaaattggagaagttcaccaattgctattctcttagcaagaccctgcgtttcaaggccattcctgtagggaaaacacaggagaatatcgataacaaacgtttgctggttgaggacgaaaagcgcgcagaagattacaagggtgtcaaaaaattgttggaccgctactatctttcgttcattaacgatgtcctgcatagtattaagcttaagaacttaaacaattacatctctttgtttcgtaagaaaacccgtactgagaaagaaaacaaggaattagagaatttggaaatcaaccttcgcaaggaaatcgcaaaggcctttaaaggaaacgagggctacaagagtctgtttaaaaaagatattatcgagacaattcttcctgagttcttggacgacaaggatgaaattgccttagtaaacagttttaatgggtttacgacggcgtttacgggcttcttcgataaccgtgagaatatgttcagcgaggaagctaagtctacgtctatcgcgttccgttgcattaatgagaatcttactcgctatattagtaacatggacattttcgagaaagtagacgcgatttttgacaaacatgaagtccaagagattaaagagaaaattttaaactcagattacgatgtggaagactttttcgaaggggagttcttcaactttgtattgacgcaggaaggaatcgatgtatataatgctattatcggcggtttcgtgactgaaagcggtgagaagattaagggtttgaatgagtatattaacctttacaatcagaaaacgaagcagaaattaccgaagttcaagccgttatataagcaagtgctgagcgatcgtgaatccctgtcgttttacggggagggatacacttctgatgaagaggtacttgaggtatttcgtaacacgttaaacaaaaactcggagattttttcctcaattaagaaactggaaaaattatttaaaaattttgatgaatactcatctgctggcatttttgttaagaacggtcccgcgatctctactattagtaaggacatttttggtgagtggaatgtaatccgtgacaaatggaacgctgaatacgatgacatccaccttaaaaagaaggctgttgttaccgagaagtacgaagacgatcgccgtaaaagctttaagaaaatcggatccttctcgttagaacaacttcaggagtacgcagacgcggatttgtctgtggttgagaagctgaaagaaattatcatccagaaggttgacgaaatttataaagtctatggaagctcggaaaagttatttgacgcggactttgttttagaaaagtcgcttaagaagaatgacgctgttgttgccattatgaaagatttattggactctgtcaaaagctttgaaaactacattaaagcctttttcggggaagggaaggaaactaatcgtgacgaatccttctacggcgactttgtattagcttatgatatccttctgaaggtggaccacatttacgatgccattcgtaactacgtgacgcagaagccctactccaaagataagtttaaattatattttcaaaaccctcagtttatgggggggtgggataaggacaaagaaacggattatcgtgctacgattttacgctatggatctaagtactatcttgccattatggataaaaaatacgcgaaatgcctgcaaaaaatcgataaggacgacgtaaacggcaactatgagaagatcaactataagttgcttcccggaccgaataagatgcttcccaaagtctttttcagtaaaaagtggatggcttactataatcccagcgaggatatccagaagatctacaaaaacggaacttttaaaaagggcgacatgttcaacttgaacgactgtcacaaacttattgacttttttaaggatagcatttcgcgctacccaaaatggagtaacgcatacgattttaatttttcagaaactgaaaagtataaggacattgccgggttctaccgcgaagtggaggagcagggatacaaggtgtcgttcgaaagcgcgagcaagaaggaagtggacaaacttgttgaagaagggaagttatatatgttccagatttataacaaggacttctccgataagtcccatggtacccctaacttgcacacgatgtacttcaagcttctgttcgacgaaaacaatcatggtcaaatccgtcttagtgggggagctgaattgttcatgcgccgcgcttcgctgaagaaagaggagttggtggttcacccagctaattcacctattgccaataagaatcccgataacccaaagaaaacgactacactttcttacgacgtatataaggacaagcgttttagcgaagatcagtatgagctgcacatcccgatcgccatcaataaatgtcccaagaatattttcaaaattaatacagaggtgcgtgtgttgttgaagcacgatgacaatccctatgtcatcggcatcgaccgtggcgaacgtaatcttttatatatcgtggttgtcgacggcaagggtaacatcgttgaacaatattccttgaatgaaatcattaataacttcaacgggattcgcattaagaccgattaccactcacttttggacaaaaaggagaaggagcgctttgaggcacgccagaactggacctctattgaaaacattaaagaacttaaggcaggatacatctcacaagttgtccacaaaatctgcgaacttgtggaaaaatacgatgccgtgatcgcgcttgaagatttaaatagcggttttaagaattctcgtgttaaggttgagaaacaggtttaccaaaagttcgaaaaaatgttaattgacaagttaaattacatggtagacaaaaaaagtaacccgtgtgccactggaggcgcactgaagggctatcagatcaccaacaaatttgaatccttcaagtcgatgagcacccagaacggctttatcttctatatcccggcatggttaacgagtaaaattgatccatcaaccggttttgtcaacttgttaaagaccaaatacacatcgattgccgactccaagaaattcatctcgtcattcgatcgcatcatgtatgtcccggaggaggatcttttcgagttcgctttagattacaagaatttttctcgtaccgacgcggattacatcaagaagtggaagttgtatagctacggtaaccgtatccgtattttccgtaatccaaaaaagaataatgtgtttgactgggaggaagtatgcttaacctcggcttacaaagagctttttaataagtacgggattaactaccagcagggggacattcgcgccctgctgtgtgagcaatccgataaagctttctatagctcattcatggcattgatgagtctgatgctgcagatgcgtaacagcatcacgggtcgcacagacgtagatttcctgatctcgcccgtgaaaaacagtgacggcatcttttatgactcgcgtaattatgaggcgcaagaaaatgccatcttgccgaaaaatgccgatgcaaacggtgcatacaacatcgcgcgtaaggtcctgtgggccattggtcaatttaaaaaagcagaagatgaaaagttggacaaagttaagatcgctattagcaacaaggagtggttagagtatgcgcagaccagcgtaaaacacgagaacttgtattttcagtcacatcatcatcaccatcatcatcaccaccatcaccaccac

SEQ ID NO: 3 - DNA, 27 bp, synthetic construct
  source 1..27: mol_type = other DNA
ttttatgtatgtgggacggtaaaccag

SEQ ID NO: 4 - DNA, 27 bp, synthetic construct
  source 1..27: mol_type = other DNA
tttatgtatgtgggacggtaaaccagg

SEQ ID NO: 5 - DNA, 27 bp, synthetic construct
  source 1..27: mol_type = other DNA
tttaccgtcccacatacataaaaaaaa

SEQ ID NO: 6 - DNA, 27 bp, synthetic construct
  source 1..27: mol_type = other DNA
ttttacatttttcgttattgctcttga

SEQ ID NO: 7 - DNA, 27 bp, synthetic construct
  source 1..27: mol_type = other DNA
tttacatttttcgttattgctcttgag

SEQ ID NO: 8 - DNA, 25 bp, synthetic construct
  source 1..25: mol_type = other DNA
tttaactatacaacctactacctca

SEQ ID NO: 9 - DNA, 21 bp, synthetic construct
  source 1..21: mol_type = other DNA
tttacaacatcagtctgataa

SEQ ID NO: 10 - RNA, 22 nt, synthetic construct
  source 1..22: mol_type = other RNA
tggtttaccgtcccacatacat

SEQ ID NO: 11 - RNA, 23 nt, synthetic construct
  source 1..23: mol_type = other RNA
tcaagagcaataacgaaaaatgt

SEQ ID NO: 12 - RNA, 22 nt, synthetic construct
  source 1..22: mol_type = other RNA
tgaggtagtaggttgtatagtt

SEQ ID NO: 13 - RNA, 22 nt, synthetic construct
  source 1..22: mol_type = other RNA
tgaggtagtaggttgtgtggtt

SEQ ID NO: 14 - RNA, 22 nt, synthetic construct
  source 1..22: mol_type = other RNA
tgaggtaggaggttgtatagtt

SEQ ID NO: 15 - RNA, 22 nt, synthetic construct
  source 1..22: mol_type = other RNA
tggagtgtgacaatggtgtttg

SEQ ID NO: 16 - DNA, 18 bp, synthetic construct
  source 1..18: mol_type = other DNA
tctacgtcggggtggttt

SEQ ID NO: 17 - DNA, 18 bp, synthetic construct
  source 1..18: mol_type = other DNA
tacgtcggggtggtttac

SEQ ID NO: 18 - DNA, 18 bp, synthetic construct
  source 1..18: mol_type = other DNA
cgtcggggtggtttaccg

SEQ ID NO: 19 - DNA, 18 bp, synthetic construct
  source 1..18: mol_type = other DNA
tctacgtcggggtcaaga

SEQ ID NO: 20 - DNA, 18 bp, synthetic construct
  source 1..18: mol_type = other DNA
tacgtcggggtcaagagc

SEQ ID NO: 21 - DNA, 18 bp, synthetic construct
  source 1..18: mol_type = other DNA
cgtcggggtcaagagcaa

SEQ ID NO: 22 - DNA, 20 bp, synthetic construct
  source 1..20: mol_type = other DNA
cgcgcaggtccagttttttt

SEQ ID NO: 23 - DNA, 22 bp, synthetic construct
  source 1..22: mol_type = other DNA
cgcgcaggtccagttttttttt

SEQ ID NO: 24 - DNA, 24 bp, synthetic construct
  source 1..24: mol_type = other DNA
cgcgcaggtccagttttttttttt

SEQ ID NO: 25 - DNA, 18 bp, synthetic construct
  source 1..18: mol_type = other DNA
tctacgtcggggtgaggt

SEQ ID NO: 26 - DNA, 20 bp, synthetic construct
  source 1..20: mol_type = other DNA
cgcgcaggtccagttttttt

SEQ ID NO: 27 - DNA, 18 bp, synthetic construct
  source 1..18: mol_type = other DNA
tacgtcggggtagcttat

SEQ ID NO: 28 - DNA, 24 bp, synthetic construct
  source 1..24: mol_type = other DNA
cgcgcaggtccagttttttttttt

SEQ ID NO: 29 - DNA, 22 bp, synthetic construct
  source 1..22: mol_type = other DNA
gcagtagcttatcagactgatg

SEQ ID NO: 30 - DNA, 26 bp, synthetic construct
  source 1..26: mol_type = other DNA
ggtccagtttttttttttttttcaac